(19) United States
(12) Patent Application Publication
(10) Pub. No.: US 2008/0057002 A1
(43) Pub. Date: Mar. 6, 2008

(54) Non-aqueous compositions containing urea and allantoin and methods for preparing same

(76) Inventor: Jerry Zhang, Grayslake, IL (US)

Correspondence Address: Jerry Zhang, 1061 Chadwick Dr., Grayslake, IL, 60030, US

(21) Appl. No.: 11/516,094

(22) Filed: Sep. 6, 2006

(51) Int. Cl. A61K 9/12; A61K 31/4166

(52) U.S. Cl. 424/45; 514/396

(57) ABSTRACT

A method for enhancing solubility of allantoin in a non-aqueous pharmaceutically acceptable alcohol carrier using a solubilization enhancer is described. Non-aqueous compositions containing the solubilized allantoin at the FDA-approved levels of 0.5% to 2% by weight are able to be obtained by using urea or urea derivatives as the solubilization enhancer. The non-aqueous topical composition is suitable for cosmetic, dermatological, and pharmaceutical use.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention concerns a method for enhancing solubility of allantoin in a non-aqueous alcohol carrier using a solubilization enhancer. A non-aqueous composition comprises a mixture of the dissolved allantoin and solubilization enhancer in the non-aqueous alcohol carrier.

### BACKGROUND OF THE INVENTION

Allantoin, known for its therapeutic action on skin, has been widely used for decades in cosmetic and over-the-counter (OTC) topical formulations. It is also used in the topical pharmaceutical applications in skin ulcer therapy, psoriasis medications and analgesic gels.

Allantoin has been classified by the Food and Drug Administration (FDA) OTC Topical Analgesic Review Panel as a Category I (safe and effective) active ingredient skin protectant at a level of 0.5% to 2%.

Although allantoin has been used extensively in cosmetic, dermatological, and pharmaceutical applications, the solubility of allantoin at the FDA-approved levels is an issue. For example, solubility of allantoin in a number of common pharmaceutical solvents is lower than the FDA-approved levels as shown in the following data (solubility expressed in g/100 g solvent@25 degree. C.): water=0.45%, ethanol <0.1%, and propylene glycol <0.1% (Solubility data are from allantoin product brochure from International Specialty Products Corp, Wayne, N.J.).

For use as a cosmetic skin protectant or treatment of many dermatological and mucosal disorders, it is often preferable to use a solubilized formulation, such as a solution or a gel, rather than a cream, lotion or an ointment. Creams, lotions, (typically oil-in-water emulsions) and ointments (typically petroleum jelly based compositions) are often comedogenic, acnegenic, or less cosmetically appealing to patients. Furthermore, active ingredient is generally more bioavailable in solubilized form than in insoluble or suspended form. Solubilized allantoin at the FDA-approved levels may be effectively used as a topical skin protectant and for treatment of dermatological and mucosal disorders. Non-aqueous allantoin compositions may be beneficial in promoting wound cleansing and healing.

Accordingly, there is a need for non-aqueous allantoin compositions where the active ingredient is solubilized not merely suspended for maximum efficacy.

### SUMMARY OF THE INVENTION

Generally speaking, in accordance with the present invention, a method is provided for enhancing solubility of allantoin in a non-aqueous alcohol carrier using a solubilization enhancer. The solubilization enhancer is a pharmaceutically acceptable organic compound.

Allantoin topical composition comprises a mixture of the solubilized allantoin and solubilization enhancer in the non-aqueous pharmaceutically acceptable alcohol carrier. The composition is in the form of a solution, a gel, or a spray.

Accordingly, it is an object of the invention to provide a method for enhancing solubility of allantoin in a non-aqueous alcohol carrier using a solubilization enhancer.

Another object of the invention is to provide a method of formulating a non-aqueous topical composition containing solubilized allantoin at a level of 0.1% by weight or more by using a solubilization enhancer.

A further object of the invention is to formulate a non-aqueous topical composition comprising a mixture of solubilized allantoin and solubilization enhancer in a non-aqueous alcohol carrier, whereby the presence of the solubilization enhancer increases the solubility of allantoin in the non-aqueous alcohol carrier when compared to the solubility of allantoin in the absence of the solubilization enhancer, and the composition is physically stable.

Still other objects and advantages of the invention will, in part, be obvious and will, in part, be apparent from the following detailed description of the preferred embodiments.

### BRIEF DESCRIPTIONS OF THE DRAWINGS

FIG. 1 shows general structure of a urea derivative.

FIG. 2 shows general structure of a mono-substituted alkyl urea.

FIG. 3 shows general structure of a mono-substituted hydroxyalkyl urea.

### DETAILED DESCRIPTION OF THE PREFERRED EMBODIMENTS

In the disclosed embodiments, a method for enhancing solubility of allantoin in a non-aqueous alcohol carrier using a solubilization enhancer is described. A non-aqueous topical composition comprises a mixture of the solubilized allantoin and solubilization enhancer in the non-aqueous alcohol carrier at allantoin level of 0.1% by weight or more.

The term ‘allantoin’, when used in accordance with the present invention, means allantoin either prepared from synthetic method or isolated from natural source, either in admixture or in pure or substantially pure form. Allantoin occurs as a tautomeric mixture, the keto and enol forms being in equilibrium. All physical forms of allantoin, crystalline, semi-crystalline, and amorphous, are contemplated and within the scope of the present invention, either in admixture or in pure or substantially pure form. Furthermore, the stereoisomers of allantoin are also contemplated and within the scope of the present invention. The definition of allantoin in accordance with the present invention embraces all possible stereoisomers and their mixtures. It particularly embraces the racemic forms and the isolated optical isomers having the specified activity.

The term ‘dissolved’, ‘dissolving’, ‘solubilized’ or ‘solubilizing’, means that allantoin or the solubilization enhancer is essentially solubilized in the non-aqueous alcohol carrier, and that allantoin or the solubilization enhancer will not exist to any appreciable degree in the particulate or crystalline form in the non-aqueous composition.

The term ‘physically stable’, when used in accordance with the present invention, means physical or solubilization stability of the composition, rather than chemical stability. The allantoin compositions are considered to be physically stable when substantially no evidence of crystal formation or precipitation is evident after they are stored at room temperature of about 18 degree. C. to about 25 degree. C. for at least 7 days.

A ‘solubilization enhancer’ is a chemical compound that when present in a solvent, increases the solubility of a second chemical compound, such as an active ingredient, in the solvent.

A ‘non-aqueous’ composition is une that is substantially water free. While water is not intentionally added to a non-aqueous composition, trace amounts of water (for example, existed in the solvent as an impurity) may still be present. It is desired that the amount of water in the composition be less than about 5% by weight, preferably less than 3% by weight, or even more preferably less than 1% by weight.

It has been unexpectedly discovered that the presence of urea or a urea derivative or any suitable combination as the solubilization enhancer increases the solubility of allantoin in the non-aqueous alcohol carrier when compared to the solubility of allantoin in the absence of such a solubilization enhancer. Physically stable, non-aqueous compositions of solubilized allantoin at a level of 0.1% by weight or more are able to be obtained by using urea or a urea derivative or any suitable combination as the solubilization enhancer. Allantoin is a polar, heterocyclic organic compound with chemical formula: C₄H₆N₄O₃ and molecular weight of 158.12 [chemical name: (2,5-dioxo-4-imidazolidinyl) urea; or 5-ureidohydantoin]. The dissolved allantoin may be present in an amount of at least 0.1% by weight, at least 0.5% by weight, at least 1% by weight, or even as much as 5% by weight. Preferably the dissolved allantoin should be present in an amount of 0.5 to 3% by weight.

It is known that polar organic compounds are likely to dissolve in polar organic solvents. Alcohols are well known polar organic solvents. Suitable alcohol solvents have the general formula of R(OH).sub.n where n is equal to or greater than 1 and R is generally C.sub.2-8 alkyl or substituted alkyl groups. Examples of suitable alcohol solvents are ethyl alcohol, 1-propanol, 2-propanol, 1-butanol, 1-pentanol, and combinations thereof.

A particular family of alcohols is polyols. For purposes of this specification, polyols, also known as polyhydric alcohols, are defined as organic compounds having at least two hydroxyl groups per molecule. The general formula of suitable polyols is: R(OH).sub.n where n is equal to or greater than 2 and R is generally C.sub.2-10 alkyl or substituted alkyl groups.

Polyols are preferred non-aqueous alcohol carriers. All stereoisomers of the polyols in accordance with the present invention are contemplated and within the scope of the present invention, either in racemic admixture or in isolated optically pure or substantially optically pure form. Examples of pharmaceutically acceptable polyols are glycerin (also known as glycerol), propylene glycol (also known as 1,2-propanediol), 1,3-propanediol, 1,2-butanediol, 1,3-butanediol, 1,4-butanediol, 2,3-butanediol, 1,2-pentanediol, 1,5-pentanediol, 1,2-hexanediol, 2-methyl-2,4,-pentanediol (also known as hexylene glycol), 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. Especially preferred polyols are glycerin, propylene glycol, 1,4-butanediol, hexylene glycol, and 1,2-hexanediol.

Another family of the suitable polyols is low molecular weight polyethylene glycol (PEG), which exists as a liquid at room temperature. Examples of pharmaceutically acceptable PEG are PEG 200, PEG 300, PEG 400, and PEG 600. The number after PEG indicates average molecular weight of a PEG.

The non-aqueous polyol carrier may be present in an amount of about 30% to about 99% by weight, more preferably about 40% to about 90% by weight.

The solubilization enhancers of the present invention are organic compounds whose presence can increase the amount of allantoin dissolved in the non-aqueous alcohol carriers when compared to the amount of allantoin that would be soluble in the non-aqueous alcohol carriers in the absence of the solubilization enhancers. Preferably the solubilization enhancers are safe, chemically stable, pharmaceutically acceptable organic compounds, or combinations of such compounds. They should also be chemically compatible with other ingredients in the non-aqueous compositions.

We have unexpectedly found that urea and urea derivatives are the suitable solubilization enhancers. Urea, a diamide of carbonic acid, is a polar organic compound. Urea has the general formula of H.sub.2 N—C(O)—NH.sub.2. Urea is widely used as a moisturizing compound or keratolytic agent in cosmetic, dermatological, and pharmaceutical applications.

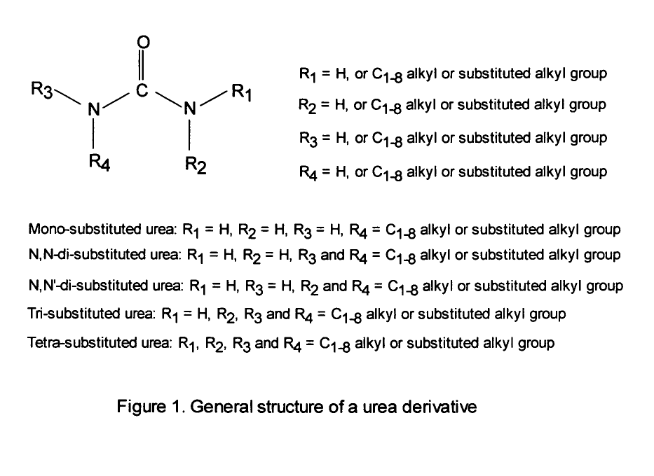

Urea derivatives are derived from urea by substituting one or more of the hydrogen atoms in the urea molecule with other suitable chemical groups. The urea derivatives, as used herein, have the general formula of R.sub.3 R.sub.4 N—C(O)—NR.sub.1 R.sub.2, where R.sub.1, R.sub.2, R.sub.3, and R.sub.4 are each independently hydrogen or C.sub.1-8 alkyl or substituted alkyl group. The alkyl group can be a straight or branched chain alkyl or a cycloalkyl group. Chemical groups such as, for example, hydroxy, can be substituted onto the alkyl chain to give the substituted alkyl group. Examples of the suitable substituted alkyl group include, but not limited to, hydroxyalkyl group. The general structure of a urea derivative is shown in FIG. 1.

When one hydrogen atom in urea molecule is substituted by an alkyl or substituted alkyl group, a mono-substituted urea is formed. When two hydrogen atoms in urea molecule are substituted by alkyl or substituted alkyl groups, a di-substituted urea is formed. There are two types of di-substituted urea: N,N-di-substituted and N,N′-di-substituted urea. When three hydrogen atoms in urea molecule are substituted by alkyl or substituted alkyl groups, a tri-substituted urea is formed. When all four hydrogen atoms in urea molecule are substituted by alkyl or substituted alkyl groups, a tetra-substituted urea is formed.

Examples of the suitable di-substituted urea are N,N-dimethyl urea, N,N′-dimethyl urea, N,N-diethyl urea, N,N′-diethyl urea, N-ethyl-N′-2-hydroxyethyl urea, N,N′-bis-(1-butyl) urea, N,N′-bis-(2-hydroxyethyl) urea, N,N-bis-(2-hydroxyethyl) urea, N,N′-bis-(3-hydroxypropyl) urea, N,N-bis-(2-hydroxypropyl) urea, N,N′-bis-(2-hydroxypropyl) urea, and N,N′-bis-(4-hydroxybutyl) urea.

Examples of the suitable tri-substituted urea are N,N-bis-(2-hydroxypropyl)-N′-(2-hydroxyethyl) urea, N,N-bis-(2-hydroxyethyl)-N′-methyl urea, N,N-bis-(2-hydroxyethyl)-N′-ethyl urea, N,N-dimethyl-N′-(2-hydroxyethyl) urea, N,N-diethyl-N′-(2-hydroxyethyl) urea, and N,N-bis-(2-hydroxyethyl)-N′-propyl urea.

Examples of the suitable tetra-substituted urea are N,N,N′,N′-tetramethyl urea, N,N,N′,N′-tetrakis-(1-butyl) urea, N,N,N′,N′-tetrakis-(2-hydroxyethyl) urea, N,N,N′,N′-tetrakis-(2-hydroxypropyl) urea, and N,N-bis-(2-hydroxyethyl)-N′,N′-dimethyl urea.

Among the substituted ureas, mono-substituted ureas are preferred solubilization enhancers. The mono-substituted ureas comprise mono-substituted alkyl and hydroxyalkyl ureas.

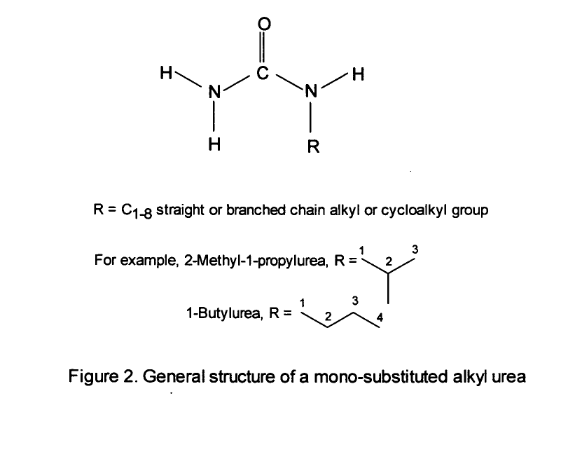

The mono-substituted alkyl ureas have the general structure as shown in FIG. 2. The alkyl group can be a straight or branched chain alkyl, or a cycloalkyl group. Examples of the suitable mono-substituted alkyl ureas are methyl urea, ethyl urea, 1-propyl urea, 2-propyl urea, 1-butyl urea, 2-butyl urea, 2-methyl-1-propyl urea, cyclohexyl urea, and combinations thereof.

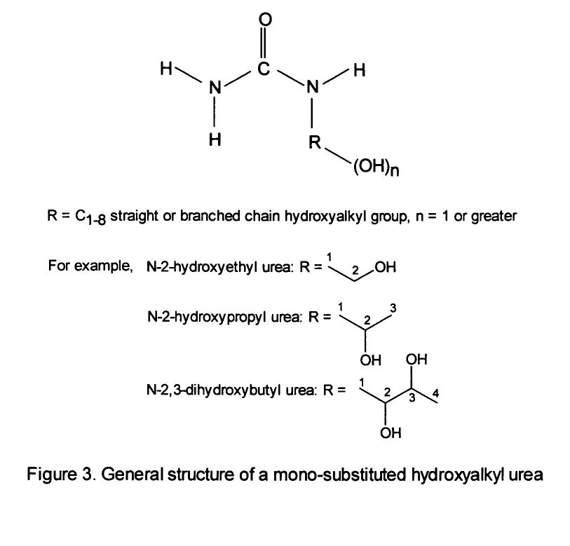

The mono-substituted hydroxyalkyl ureas have the general structure as shown in FIG. 3. The alkyl group can be a straight or branched chain alkyl group with one or more hydroxyl groups attached onto the alkyl chain at any suitable positions. Examples of the suitable mono-substituted hydroxyalkyl ureas are N-2-hydroxyethyl urea, N-3-hydroxypropyl urea, N-2-hydroxypropyl urea, N-2,3-dihydroxypropyl urea, N-4-hydroxybutyl urea, N-3-hydroxybutyl urea, N-2-hydroxybutyl urea, N-2,3-dihydroxybutyl urea, N-2,4-dihydroxybutyl urea, N-3,4-dihydroxybutyl urea, and combinations thereof.

Combinations of urea, alkyl ureas, or hydroxyalkyl ureas in any given ratio are also suitable as the solubilization enhancers. The mono-substituted alkyl ureas are more hydrophobic than the parent compound urea due to the alkyl group. The hydrophobic property of the solubilization enhancers might be adjusted by varying the amount of the mono-substituted alkyl ureas being combined with urea.

Urea is most preferred solubilization enhancer.

The dissolved urea or urea derivatives may be present in an amount of at least 1% by weight, at least 5% by weight, at least 10% weight, or even as much as 50% by weight. Preferably the dissolved solubilization enhancers should be present in an amount of about 5% to about 40% by weight.

In one embodiment where the solubilization enhancer is dissolved first before allantoin is added, the method for dissolving allantoin in the non-aqueous alcohol solvent using the solubilization enhancer is as follows:
heating the alcohol solvent to a temperature of about 40 degree. C. to about 150 degree. C., preferably to a temperature of about 50 degree. C. to about 100 degree. C.,
adding the solubilization enhancer to the heated solvent while maintaining the desirable temperature, while stirring until dissolved,
adding allantoin to the heated solvent containing the dissolved solubilization enhancer while maintaining the desirable temperature, while stirring until dissolved,
cooling the mixture to room temperature.

In another embodiment where the solubilization enhancer and allantoin are added together, the method for dissolving allantoin in the non-aqueous alcohol solvent using the solubilization enhancer is as follows:
heating the alcohol solvent to a temperature of about 40 degree. C. to about 150 degree. C., preferably to a temperature of about 50 degree. C. to about 100 degree. C.,
adding the solubilization enhancer and allantoin together to the heated solvent while maintaining the desirable temperature, while stirring until dissolved,
cooling the mixture to room temperature.

In yet another embodiment, the solubilization enhancer can be added to the non-aqueous alcohol solvent at room temperature. The mixture is then heated to a temperature of about 40 degree. C. to about 150 degree. C., preferably to a temperature of about 50 degree. C. to about 100 degree. C., while stirring until dissolved. Next, allantoin was added to the heated mixture while maintaining the desirable temperature, while stirring until dissolved. Then, the mixture is cooled to room temperature.

In still another embodiment, the solubilization enhancer and allantoin can be added together to the non-aqueous alcohol solvent at room temperature. The mixture is then heated to a temperature of about 40 degree. C. to about 150 degree. C., preferably to a temperature of about 50 degree. C. to about 100 degree. C., while stirring until dissolved. Then, the mixture is cooled to room temperature.

It has been unexpectedly discovered that the method of cooling the mixture containing the dissolved solubilization enhancer and allantoin is important in promoting the physical stability of the prepared compositions. Cooling the heated mixture can be achieved by either regular cooling or rapid cooling. Although regular cooling to room temperature can produce physically stable compositions, rapid cooling yields more stable compositions, especially at higher allantoin concentrations. “Regular cooling” means that the heated mixture is removed from the heat source and exposed to ambient temperature. “Rapid cooling” means that the heated mixture is cooled to room temperature by putting it in an ice bath. The preferred method of cooling is rapid cooling in ice bath.

It has also been discovered that reheating the cooled mixture back to the desired temperature and then cooling it again can also enhance the physical stability of the prepared compositions. A “reheating and cooling cycle” means that after a heated mixture is cooled to room temperature by either regular cooling or rapid cooling, the cooled mixture is heated again to the desired temperature and then it is cooled again back to room temperature. This reheating and cooling cycle can be repeated a number of times if needed, until the desired physical stability of the prepared compositions are achieved.

The composition, in accordance with the present invention, may be in the form of a gel, a solution, or a spray. Preferably the composition is a gel. Therefore, the non-aqueous allantoin composition preferably contains a gelling agent. Any gelling agent that is dispersible in the non-aqueous alcohol solvents and forms a non-aqueous gel of substantially uniform consistency is suitable for use in the present invention. The gelling agents should not substantially decrease the solubility of allantoin in the non-aqueous alcohol solvent or reduce the therapeutic efficacy of the composition. “Substantially decrease” means that the inclusion of the gelling agent decreases the solubility of allantoin to 0.1% by weight or less in the composition. Examples of the suitable gelling agents are: polycarbohydrate gelling agents and carbomers. Examples of the suitable polycarbohydrate gelling agents are hydroxyethylcellulose, hydroxypropylcellulose, and xanthan gum. Examples of the suitable carbomers are CARBOPOL Brand 934, 940, 941, Ultrez 10, and Ultrez 20 from Noveon Corp., Cleveland, Ohio. Combinations of the polycarbohydrate gelling agents and carbomers are also suitable as the gelling agents.

Carbomers are high molecular weight homo- and copolymers of acrylic acid crosslinked with a polyalkenyl polyether. They need to be neutralized by basic neutralizing agents to achieve their rheological features and performance properties. Since allantoin and urea degrade in basic conditions (i.e., chemically unstable), the pH value of the non-aqueous composition after neutralization should be 6.5 or less, more preferably 6.0 or less, even more preferably 5.5 or less. Organic base compounds are preferred neutralizing agents. Examples of the suitable organic base are aminoethyl propanol, triethanol amine, diisopropanol amine, triisopropanol amine, and tetrahydroxypropyl ethylenediamine.

The non-aqueous allantoin composition of the present invention can contain conventional amounts of one or more other desirable ingredients: vitamins, coenzymes, skin penetration enhancers, emulsifiers, emollients, herbal extracts, chelating agents, antibiotics, colorants, antioxidants, and even sunscreens. Examples of the suitable desirable ingredients are: ascorbic acid (vitamin C), grape seed extract, tocopherols (vitamin E), tocopherol acetate, tocopherol succinate, retinol, retinoic acids, vitamin A palmitate, vitamin K and derivatives, silicone-polyol copolymer emulsifiers, or butylated hydroxytoluene. They may be present in an amount of at least 0.01% by weight, 1% by weight, or even as much as 10% by weight.

The following examples are included for purposes of illustrating the technology covered by this disclosure. They are not intended to limit the scope of the claimed invention in any manner. One skilled in the art will understand that there are alternatives to these specific embodiments that are not completely described by these examples.

### EXAMPLE 1

This example is to demonstrate the solubilization enhancing effect of urea in propylene glycol at 5% urea concentration.

| Component | Amount (weight percentage) |
|---|---|
| Allantoin | 0.5% |
| Urea | 5% |
| Propylene glycol | 94.5% |

Propylene glycol was heated to about 65 degree. C. Allantoin was added to the propylene glycol at 65 degree. C. The mixture was maintained at 65 degree. C., while stirring until dissolved. The sample was allowed to cool to room temperature. It was a clear solution and physically stable for at least 7 days.

### EXAMPLE 2

This example is to demonstrate the solubilization enhancing effect of urea in propylene glycol at 1% allantoin concentration. A non-aqueous composition in accordance with the invention was prepared as follows:

| Component | Amount (weight percentage) |
|---|---|
| Allantoin | 1% |
| Urea | 25% |
| Propylene glycol | 74% |

Propylene glycol was heated to 65 degree. C. Urea and allantoin were added together to the propylene glycol while maintaining the temperature at 65 degree. C., while stirring until dissolved. The sample was allowed to cool to room temperature. It was a clear solution and physically stable for at least 7 days.

### EXAMPLE 3

This example is to demonstrate the solubilization enhancing effect of urea in propylene glycol at 2% allantoin concentration. A non-aqueous composition in accordance with the invention was prepared as follows:

| Component | Amount (weight percentage) |
|---|---|
| Allantoin | 2% |
| Urea | 25% |
| Propylene glycol | 73% |

Propylene glycol was heated to 65 degree. C. Urea and allantoin were added together to the propylene glycol while maintaining the temperature at 65 degree. C., while stirring until dissolved. The sample was then put into an ice bath until the sample was cooled to room temperature. Then, a second reheating and cooling cycle was performed. The final product was a clear solution and physically stable for at least 7 days.

### EXAMPLE 4

This example is to demonstrate the solubilization enhancing effect of methyl urea, a mono-substituted alkyl urea, in propylene glycol. A non-aqueous composition in accordance with the invention was prepared as follows:

| Component | Amount (weight percentage) |
|---|---|
| Allantoin | 1% |
| Methyl urea | 25% |
| Propylene glycol | 74% |

Propylene glycol was heated to 65 degree. C. Methyl urea was added to the propylene glycol while maintaining the temperature at 65 degree. C., while stirring until dissolved. Methyl urea is available from Lancaster Synthesis (Ward Hill, Mass.). Then, allantoin was added to the mixture while maintaining the temperature at 65 degree. C., while stirring until dissolved. The sample was allowed to cool to room temperature. The sample was a clear solution and physically stable for at least 7 days.

### EXAMPLE 5

This example is to demonstrate the solubilization enhancing effect of N-2-hydroxyethyl urea, a mono-substituted hydroxyalkyl urea, in propylene glycol. A non-aqueous composition in accordance with the invention was prepared as follows:

| Component | Amount (weight percentage) |
|---|---|
| Allantoin | 1% |
| N-2-hydroxyethyl urea | 16% |
| Propylene glycol | 83% |

Propylene glycol was heated to 65 degree. C. N-2-hydroxyethyl urea was added to the propylene glycol while maintaining the temperature at 65 degree. C., while stirring until dissolved. N-2-hydroxyethyl urea is available from Aldrich Chemical Company (Milwaukee, Wis.). Then, allantoin was added to the mixture while maintaining the temperature at 65 degree. C., while stirring until dissolved. The sample was allowed to cool to room temperature. The sample was a clear solution and physically stable for at least 7 days.

### EXAMPLE 6

This example is to demonstrate the solubilization enhancing effect of urea in propylene glycol and the composition is in the form of a gel. A non-aqueous composition in accordance with the invention was prepared as follows:

| Component | Amount (weight percentage) |
|---|---|
| Allantoin | 1% |
| Urea | 25% |
| Propylene glycol | 73.5% |
| Ultrez 10 | 0.5% |

Propylene glycol was heated to 65 degree. C. Urea and allantoin were added together to the propylene glycol while maintaining the temperature at 65 degree. C., while stirring until dissolved. The sample was allowed to cool to room temperature. Then, a carbomer thickener, Ultrez 10 (available from Noveon Corp., Cleveland, Ohio), was dispersed into the solution in a shifted fashion. After Ultrez 10 was completely dispersed, the mixture was neutralized by triethanol amine to pH about 5.5. The fmal product was a clear gel of uniform consistency and physically stable for at least 7 days.

### EXAMPLE 7

This example is to demonstrate the solubilization enhancing effect of urea in a mixture of propylene glycol and PEG 400. A non-aqueous composition in accordance with the invention was prepared as follows:

| Component | Amount (weight percentage) |
|---|---|
| Allantoin | 1.5% |
| Urea | 22% |
| PEG 400 | 10% |
| Propylene glycol | 66.5% |

The mixture of propylene glycol and PEG 400 was heated to 65 degree. C. Urea and allantoin were added together to the mixed solvent while maintaining the temperature at 65 degree. C., while stirring until dissolved. Then, the sample was put into an ice bath until the sample was cooled to room temperature. The sample was subject to second heating cycle. The sample was again heated to 65 degree. C. Then, it was subject to second rapid cooling cycle by putting it in an ice bath to cool to room temperature. The final product was a clear solution and physically stable for at least 7 days.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible mixtures of such ingredients wherever the sense permits.

What is claimed is:

1. A method for enhancing solubility of allantoin in a non-aqueous alcohol solvent using a solubilization enhancer selected from the group consisting of urea, urea derivatives, and combinations thereof, comprising:
(a) placing the solubilization enhancer and allantoin in intimate contact with the non-aqueous alcohol solvent,
(b) subjecting the non-aqueous alcohol solvent containing the solubilization enhancer and allantoin to a temperature of about 40 degree. C. to about 150 degree. C. for sufficient time to permit the solubilization enhancer and allantoin to dissolve in the non-aqueous alcohol solvent,
(c) cooling the mixture to room temperature,
(d) optionally, performing a reheating and cooling cycle at least once until the cooled mixture achieves the desired physical stability,
whereby the amount of allantoin solubilized in the cooled mixture containing the dissolved solubilization enhancer is greater than the amount of allantoin that would be soluble in the non-aqueous alcohol solvent in the absence of the solubilization enhancer, and the mixture is physically stable.

2. The method of claim 1 wherein the solubility of allantoin is increased to about 0.1% by weight or more.

3. The method of claim 1 wherein the solubility of allantoin is increased to about 0.5% by weight or more.

4. The method of claim 1 wherein the non-aqueous alcohol solvent comprises polyol.

5. The method of claim 1 wherein the solubilization enhancer is urea.

6. The method of claim 1 wherein the solubilization enhancer comprises mono-substituted hydroxyalkyl urea.

7. The method of claim 1 wherein the solubilization enhancer comprises mono-substituted alkyl urea.

8. The method of claim 1 wherein the mixture is cooled by rapid cooling.

9. The method of claim 1 wherein the optional step (d) is conducted at least once.

10. A method for preparing a non-aqueous composition containing solubilized allantoin at a level of 0.1% by weight or more, comprising:
(a) combining allantoin, a solubilization enhancer selected from the group consisted of urea, urea derivatives, and combinations thereof, in an amount of about 1% to about 50% by weight, in a non-aqueous alcohol solvent,
(b) subjecting the mixture to a temperature of about 40 degree. C. to about 150 degree. C. for sufficient time to permit the solubilization enhancer and allantoin to dissolve in the non-aqueous alcohol solvent,
(c) cooling the mixture to room temperature,
(d) optionally, performing a reheating and cooling cycle at least once until the cooled mixture achieves the desired physical stability,
whereby the amount of allantoin solubilized in the cooled mixture containing the dissolved solubilization enhancer is greater than the amount of allantoin that would be soluble in the non-aqueous alcohol solvent in the absence of the solubilization enhancer, and the mixture is physically stable.

11. The method of claim 10 wherein the non-aqueous alcohol solvent comprises polyol.

12. The method of claim 10 wherein the solubilization enhancer is urea.

13. The method of claim 10 wherein the non-aqueous composition is in the form of a gel, a solution, or a spray.

14. A non-aqueous composition comprising, by weight of the total composition:
allantoin, in an amount of about 0.1% to about 5%,
a solubilization enhancer selected from the group consisting of urea, urea derivatives, and combinations thereof, in an amount of about 1% to about 50%,
a non-aqueous alcohol solvent, in an amount of about 30% to about 99%,
whereby the amount of allantoin solubilized in the non-aqueous composition containing the dissolved solubilization enhancer is greater than the amount of allantoin that would be soluble in the absence of the solubilization enhancer, the solubilization enhancer and allantoin are essentially solubilized in the non-aqueous composition, and the composition is physically stable.

15. The composition of claim 14 wherein the solubilization enhancer comprises mono-substituted hydroxyalkyl urea.

16. The composition of claim 14 wherein the solubilization enhancer comprises mono-substituted alkyl urea.

17. The composition of claim 14 wherein the non-aqueous alcohol solvent comprises polyol.

18. The composition of claim 14 wherein the solubilization enhancer is urea.

19. The composition of claim 14 which is in the form of a solution, a gel, or a spray.

20. The composition of claim 14 which is a gel.
